(19) United States
(12) Patent Application Publication
(10) Pub. No.: US 2005/0054869 A1
(43) Pub. Date: Mar. 10, 2005

(54) Mixed metal oxide catalysts for propane and isobutane oxidation and ammoxidation, and methods of preparing same

(76) Inventors: Claus G. Lugmair, San Jose, CA (US); Jessica Zysk, Chicago, IL (US); Robert Karl Grasselli, Chadds Ford, PA (US)

Correspondence Address: BP America Inc.;BP Legal, Mail Code 5 East, 4101 Winfield Road, Warrenville, IL, 60555, US

(21) Appl. No.: 10/860,369

(22) Filed: Jun. 3, 2004

Related U.S. Application Data
(60) Provisional application No. 60/476,528, filed on Jun. 6, 2003.
(60) Provisional application No. 60/486,433, filed on Jul. 14, 2003.

(51) Int. Cl. C07C 253/26; C07C 51/16

(52) U.S. Cl. 558/323; 502/312; 562/547

(57) ABSTRACT

Compositions of matter and catalyst compositions effective for gas-phase conversion of propane to acrylic acid (via oxidation) or to acrylonitrile (via ammoxidation) and isobutane to methacrylic acid (via oxidation) and isobutane to methacrylonitrile (via ammoxidation) are disclosed. Preferred catalyst compositions comprise molybdenum, vanadium, niobium, antimony and germanium and molybdenum, vanadium, tantalum, antimony, and germanium. Methods of preparing such compositions and related compositions, including hydrothermal synthesis methods are also disclosed. The preferred catalysts convert propane to acrylic acid and/or to acrylonitrile and isobutane to methacrylic acid/methacrylonitrile with a yield of at least about 50%.

This application claims the benefit of U.S. Provisional Application 60/476,528 filed Jun. 6, 2003 and U.S. Provisional Application 60/486,433 filed Jul. 14, 2003.

### BACKGROUND OF THE INVENTION

The present invention generally relates to compositions of matter, catalyst compositions, methods of preparing such compositions of matter and such catalyst compositions, and methods of using such compositions of matter and such catalyst compositions. Preferably, in each case, such compositions and such catalysts are effective for gas-phase conversion of propane to acrylic acid and isobutane to methacrylic acid (via oxidation) or of propane to acrylonitrile and isobutene to methacrylonitrile (via ammoxidation), and most preferably with a yield of at least about 50%.

The invention particularly relates, in a preferred embodiment, to compositions of matter, catalyst compositions, methods of preparing such compositions of matter and such catalyst compositions, and methods of using such compositions of matter and such catalyst compositions, where in each case, the same comprises molybdenum, vanadium, niobium and antimony; or molybdenum, vanadium, tantalum and antimony, and in some embodiments, each further comprises germanium. Preferred embodiments for preparing such compositions of matter and catalyst compositions include reactions in solution phase in sealed reaction vessels at temperatures above 100° C. and at pressures above ambient pressure. Hydrothermal synthesis using aqueous solutions is particularly preferred.

Generally, the field of the invention relates to molybdenum-containing and vanadium-containing catalysts shown to be effective for conversion of propane to acrylic acid (via an oxidation reaction) and/or for conversion of propane to acrylonitrile (via an ammoxidation reaction). The art known in this field includes numerous patents and patent applications, including for example, U.S. Pat. No. 6,043,185 to Cirjak et al., U.S. Pat. No. 6,514,902 to Inoue et al., U.S. Pat. No. 6,143,916 to Hinago et al., U.S. Pat. No. 6,383,978 to Bogan, Jr., U.S. Patent Application No. US 2002/0115879 A1 by Hinago et al., U.S. Patent Application No. 2003/0004379 to Gaffney et al., Japanese Patent Application No. JP 1999/114426 A by Asahi Chemical Co., Japanese Patent Application No. JP 2002/191974 A by Asahi Chemical Co., PCT Patent Application No. WO 01/98246 A1 by BASF A.G., as well as numerous literature publications, including for example, Watanabe et al., “New Synthesis Route for Mo—V—Nb—Te mixed oxides catalyst for propane ammoxidation”, Applied Catalysis A: General, 194-195, pp. 479-485 (2000), and Ueda et al., “Selective Oxidation of Light Alkanes over hydrothermally synthesized Mo—V—M—O (M=Al, Ga, Bi, Sb and Te) oxide catalysts”, Applied Catalysis A: General, 200, pp. 135-145.

Although advancements have been made in the art connection with molybdenum-containing and vanadium-containing catalysts effective for conversion of propane to acrylic acid and isobutane to methacrylic acid (via an oxidation reaction) and/or for conversion of propane to acrylonitrile and isobutane to methacrylonitrile (via an ammoxidation reaction), the catalysts need further improvement before becoming commercially viable. In general, the art-known catalytic systems for such reactions suffer from generally low yields of the desired product. Also, the synthesis protocols known in the art for such catalyst systems are difficult to reproduce in a manner that leads to consistency in catalyst performance.

### SUMMARY OF INVENTION

It is therefore an object of the present invention to overcome the above-noted deficiencies of the prior art catalyst compositions.

It is also an object of the invention to provide catalysts having improved yield in connection with the gas-phase oxidation and/or ammoxidation of propane to form acrylic acid and/or acrylonitrile, respectively and the gas-phase oxidation and/or ammoxidation of isobutane to form methacrylic acid and/or methacrylonitrile, respectively. It is a further object of the invention to provide methods of preparing catalysts that reproducibly lead to consistent catalytic performance.

Briefly, therefore, the present invention is directed to the subject matter defined by the claims hereof, as well as the subject matter disclosed herein, specifically including the various combinations and permutations that would be known to those of skill in the art based on the teaching herein.

The compositions of matter, the catalyst compositions, the methods for preparing the catalysts, the catalysts prepared by such methods, the methods of using such catalysts each offer advantages over known such systems. Uses of such catalysts include bench scale (R&D), pilot plant scale and commercial scale reaction systems for converting propane as a feedstock to acrylic acid via oxidation or to acrylonitrile via ammoxidation. The catalyst may also be used on the same scales and in the same systems to convert isobutane to methacrylic acid and/or methacrylonitrile.

Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

FIGS. 1A and 1B are schematic representations of exemplary propane and isobutane oxidation reactions (FIG. 1A) and exemplary propane and isobutane ammoxidation reactions (FIG. 1B).

### DETAILED DESCRIPTION OF THE INVENTION

Compositions of Matter and Catalyst Compositions

In one first aspect, the present invention is directed to compositions that comprise molybdenum, vanadium, niobium, antimony, germanium, and oxygen; or molybdenum, vanadium, tantalum, antimony, germanium, and oxygen.

In another second aspect, the invention is directed to compositions that are catalysts comprising a mixed metal oxide effective for vapor phase conversion of propane to acrylic acid and/or acrylonitrile and/or isobutane to methacrylic acid and/or methacrylonitrile. The mixed metal oxide has a composition comprising molybdenum, vanadium, niobium, antimony, germanium, and oxygen; or molybdenum, vanadium, tantalum, antimony, germanium, and oxygen. Preferably, the mixed metal oxide has an empirical formula
Mo₁VₐNb_{b}Sb_{c}Ge_{d}Oₓ or Mo₁VₐTa_{b}Sb_{c}Ge_{d}Oₓ,
wherein,
a ranges from about 0.1 to about 0.6, preferably from about 0.15 to about 0.5, and most preferably from about 0.2 to about 0.4, and is particularly preferred as being about 0.3,
b ranges from about 0.02 to about 0.12, preferably from about 0.03 to about 0.1, and most preferably from about 0.04 to about 0.08, and is particularly preferred as being about 0.06,
c ranges from about 0.1 to about 0.5, preferably from about 0.15 to about 0.35, more preferably from about 0.15 to about 0.3, and most preferably from about 0.2 to about 0.3, and is particularly preferred as being about 0.2,
d ranges from about 0.01 to about 1, in one embodiment the lower end of the d range is about 0.05, in another embodiment the lower end of the d range is about 0.1, in another embodiment to lower end of the d range is greater than 0.1, in another embodiment the lower end of the d range is about 0.15, in yet another embodiment the lower end of the d range is about 0.2, in yet another embodiment the lower end of the d range is greater than 0.2; in one embodiment the upper end of the d range is about 0.7, in another embodiment the upper end of the d range is about 0.5, in yet another embodiment d ranges from about 0.2 to about 0.4, and is particularly preferred as being about 0.3, and
x depends on the oxidation state of other elements present in the mixed metal oxide.

In a further third aspect of the invention, the invention is directed to the first or second aspects of the invention as described above, and further comprising an essential absence of one or more of tellurium, cerium and/or gallium, in various permutations and combinations. With respect to the essential absence of tellurium, it has been discovered that catalysts comprising molybdenum, vanadium, niobium and the combination of antimony and germanium are more active, with respect to the conversion of propane to acrylonitrile, than catalysts comprising molybdenum, vanadium, niobium and the combination of tellurium and germanium.

In a still further fourth aspect of the invention, the invention is directed to a composition of matter or to a catalyst comprising a mixed metal oxide, such as to the first or second aspects of the invention as described above, where the composition of matter or the catalyst comprising a mixed metal oxide, in each case consists essentially of molybdenum, vanadium, niobium, antimony, germanium, and oxygen or molybdenum, vanadium, tantalum, antimony, germanium, and oxygen.

In any of the aforementioned first through fourth aspects of the invention, the composition of matter can have stoichiometric ratios of the required elements relative to each other. The stoichiometric ratios can express the relative atomic ratios or molar ratios within the material (e.g., on average), or alternatively, at least a portion of the material (e.g., in one phase of a two-phase system). For example, the ratio of molybdenum to vanadium ranges from about 1:0.1 to about 1:0.6, preferably from about 1:0.15 to about 1:0.5, and most preferably from about 1:0.2 to about 1:0.4. The ratio of molybdenum to niobium or molybdenum to tantalum ranges from about 1:0.02 to about 1:0.12, preferably from about 1:0.03 to about 1:0.1, and most preferably from about 1:0.04 to about 1:0.06. The ratio of molybdenum to antimony ranges from about 1:0.1 to about 1:0.5, preferably from about 1:0.15 to about 1:0.35, more preferably from about 1:0.15 to about 1:0.3, and most preferably from about 1:0.2 to about 1:0.3. The ratio of molybdenum to germanium ranges from about 1:0.01 to about 1:1, preferably from about 1:0.05 to about 1:1, still preferably from about 1:0.1 to about 1:1, more preferably from about 1:0.1 to about 1:0.7, even more preferably from about 1:0.1 to about 1:0.5, and most preferably from about 1:0.2 to about 1:0.4. In another embodiment, the ratio of molybdenum to germanium ranges from 1:>0.1 to about 1:1. In yet another embodiment the ratio of molybdenum to germanium ranges from 1:0.15 to about 1:1. In another embodiment, the ratio of molybdenum to germanium ranges from 1:>0.2 to about 1:1. It will be appreciated that each of the preferred ranges for each of the components can be combined in various permutations and combinations.

Expressed as in the second aspect of the invention, the stoichiometric ratios of the components can be defined in connection with the empirical formula, wherein, the mixed metal oxide has an empirical formula Mo₁VₐNb_{b}Sb_{c}Ge_{d}Oₓ, or Mo₁VₐTa_{b}Sb_{c}Ge_{d}Oₓ, wherein a, b, c, d and x have preferred ranges as described above in connection with the second aspect of the invention.

Hence, a first preferred catalyst composition comprises a mixed metal oxide, Mo₁VₐNb_{b}Sb_{c}Ge_{d}Oₓ or Mo₁VₐTa_{b}Sb_{c}Ge_{d}Oₓ, where a ranges from about 0.1 to about 0.6, b ranges from about 0.02 to about 0.12, c ranges from about 0.1 to about 0.5, d ranges from about 0.01 to about 1, in another embodiment d ranges from greater than 0.1 to about 1, in yet another embodiment d ranges from greater than 0.2 to about 1, and x depends on the oxidation state of other elements present in the mixed metal oxide.

A second preferred catalyst composition comprises a mixed metal oxide, Mo₁VₐNb_{b}Sb_{c}Ge_{d}Oₓ or Mo₁VₐTa_{b}Sb_{c}Ge_{d}Oₓ, where a ranges from about 0.15 to about 0.5, b ranges from about 0.03 to about 0.1, c ranges from about 0.15 to about 0.35, d ranges from about 0.05 to about 1, in another embodiment d ranges from greater than 0.1 to about 1, in yet another embodiment d ranges from greater than 0.2 to about 1, and x depends on the oxidation state of other elements present in the mixed metal oxide.

A third preferred catalyst composition comprises a mixed metal oxide, Mo₁VₐNb_{b}Sb_{c}Ge_{d}Oₓ or Mo₁VₐTa_{b}Sb_{c}Ge_{d}Oₓ, where a ranges from about 0.2 to about 0.4, b ranges from about 0.04 to about 0.08, c ranges from about 0.15 to about 0.3, d ranges from about 0.1 to about 0.7, preferably greater than 0.1 to about 0.7, in another embodiment d ranges from about 0.2 to about 1, preferably greater than 0.2 to about 0.7, and x depends on the oxidation state of other elements present in the mixed metal oxide.

Preparation of Catalyst Compositions

The compositions and catalysts defined by the aforementioned first through fourth aspects of the invention can be prepared by the hydrothermal synthesis methods described herein. However, since such methods themselves define independent aspects of the invention, such additional aspects of the invention can be effectively applied to prepare other compositions and catalysts, including compositions and catalysts that are more broadly characterized.

Hence, for example, a fifth aspect of the invention is directed towards a hydrothermal synthesis method for preparing mixed metal oxide composition and in a preferred aspect a catalyst comprising a mixed metal oxide containing molybdenum, vanadium, niobium and antimony or molybdenum, vanadium, tantalum, antimony, germanium, and oxygen, discussed below. Hydrothermal synthesis methods are disclosed in U.S. Patent Application No. 2003/0004379 to Gaffney et al., Watanabe et al., “New Synthesis Route for Mo—V—Nb—Te mixed oxides catalyst for propane ammoxidation”, Applied Catalysis A: General, 194-195, pp. 479-485 (2000), and Ueda et al., “Selective Oxidation of Light Alkanes over hydrothermally synthesized Mo—V—M—O (M=Al, Ga, Bi, Sb and Te) oxide catalysts.”, Applied Catalysis A: General, 200, pp. 135-145, which are incorporated here by reference. Accordingly, the invention includes an improved hydrothermal synthesis where precursors for a mixed metal oxide compound are admixed in an aqueous solution to form a reaction medium and reacting the reaction medium at elevated pressure and elevated temperature in a sealed reaction vessel for a time sufficient to form the mixed metal oxide. The improvement in the method is the agitation of the reaction medium during the reaction step. Agitating the reaction medium, as discussed below, may be accomplished by a number of means such as stirring within the reaction vessel, or, for example, tumbling, shaking or vibrating the reaction vessel. Agitating the reaction mixture during the reaction step provides a number of advantages. This improvement provides more uniform mixing during the reaction, particularly with marginally soluble reactants. This results in more efficient consumption of starting materials and in a more uniform mixed metal oxide product. Agitating the reaction medium during the reaction step also causes the mixed metal oxide product to from in solution rather than on the sides of the reaction vessel. This allows more ready recovery and separation of the mixed metal oxide product by techniques such as centrifugation, decantation, or filtration and avoids the need to recover the majority of product from the sides of the reactor vessel. See U.S. Application 2003/0004379 A1 where the product of the hydrothermal synthesis formed on the reactor vessel walls. More advantageously, having the mixed metal oxide form in solution allows for particle growth on all faces of the particle rather than the limited exposed faces when the growth occurs out from the reactor wall.

This fifth aspect of the invention can be also directed more broadly, for example, toward preparing a catalyst comprising a mixed metal oxide comprising at least two of molybdenum, vanadium, antimony and tellurium, and preferably comprising at least molybdenum and vanadium, or comprising at least molybdenum and antimony, or comprising at least vanadium and antimony. Optionally, in each of such cases of this fifth aspect of the invention, the method can be directed toward preparing a catalyst comprising a mixed metal oxide that further comprises one or more of niobium, tantalum, germanium and/or other elements known in the art in combination with such systems.

According to the fifth aspect, the invention relates to a method for preparing a mixed metal oxide comprising molybdenum, vanadium, niobium, and antimony or molybdenum, vanadium, tantalum, antimony, germanium, and oxygen. The method:
admixes, in a reaction vessel, precursor compounds of Mo, V, Nb or Ta, and Sb in an aqueous solvent to form a reaction medium having an initial pH of 4 or less;
optionally adds additional aqueous solvent to the reaction vessel;
seals the reaction vessel;
reacts the reaction medium at a temperature greater than 100° C. and a pressure greater than ambient pressure for a time sufficient to form a mixed metal oxide;
optionally cooling the reaction medium; and
recovering the mixed metal oxide from the reaction medium.
   Another method according to the fifth aspect of the invention prepares a mixed metal oxide comprising molybdenum, vanadium, niobium, and antimony or molybdenum, vanadium, tantalum, antimony, and oxygen by:
admixing, in a reaction vessel, precursor compounds of Mo, V, Nb or Ta, and Sb in an aqueous solvent to form a reaction medium;
optionally adding additional aqueous solvent to the reaction vessel;
sealing the reaction vessel;
reacting the reaction medium at a temperature greater than 100° C. and a pressure greater than ambient pressure while agitating the reaction medium for a time sufficient to form a mixed metal oxide;
optionally cooling the reaction medium; and
recovering the mixed metal oxide from the reaction medium.
   When the mixed metal oxide contains germanium, the admixing step further comprises admixing a compound of Ge.

A sixth aspect of the invention is directed towards preparing a catalyst comprising a mixed metal oxide and having the empirical formula Mo₁VₐNb_{b}Sb_{c}Oₓ or Mo₁VₐTa_{b}Sb_{c}Oₓ, where component a ranges from about 0.1 to about 0.6, preferably from about 0.15 to about 0.5, and most preferably from about 0.2 to about 0.4, where component b ranges from about 0.02 to about 0.12, preferably from about 0.03 to about 0.1, and most preferably from about 0.04 to about 0.08, and where component c ranges from about 0.1 to about 0.5, preferably from about 0.15 to about 0.35, more preferably from about 0.15 to about 0.3, and most preferably from about 0.2 to about 0.3. This sixth aspect of the invention can be also directed more broadly, toward preparing a catalyst comprising a mixed metal oxide having the empirical formula Mo₁VₐX_{b}Y_{c}Oₓ, where X is optional, but can be preferably selected from niobium or tantalum, Y is optional, but can be preferably selected from antimony and tellurium, and component a ranges from about 0.1 to about 0.6, preferably from about 0.15 to about 0.5, and most preferably from about 0.2 to about 0.4, where component b ranges from 0 to about 0.12, preferably from about 0.02 to about 0.12, more preferably from about 0.03 to about 0.1, and most preferably from about 0.04 to about 0.08, and where component c ranges from 0 to about 0.5, preferably from about 0.1 to about 0.5, more preferably from about 0.15 to about 0.35, more preferably from about 0.15 to about 0.3, and most preferably from about 0.2 to about 0.3, and x depends on the oxidation state of the other elements present in the mixed metal oxide.

A seventh aspect of the invention is directed towards preparing a catalyst comprising a mixed metal oxide as defined in the fifth and sixth aspects of the invention, and further comprising germanium. More specifically, expressed in terms of an empirical formula, the catalyst can comprise a mixed metal oxide having the empirical formula Mo₁VₐNb_{b}Sb_{c}Ge_{d}Oₓ or Mo₁VₐTa_{b}Sb_{c}Ge_{d}Oₓ, where a, b, c and d have values as described above in connection with the second aspect of this invention, including ranges of preferred compositions within such described ranges, and x depends on the oxidation state of other elements present in the mixed metal oxide.

In any of the fifth, sixth or seventh aspects of the invention, the hydrothermal synthesis method can comprise several steps, as described both generally and specifically above and hereinafter.

Among these steps is included the step of forming an aqueous liquid reaction medium (e.g., as a solution, as a uniform or non-uniform dispersion, such as a slurry, or as a combination of both a solution and a dispersion), where the liquid reaction medium comprises the required components in the reaction vessel—for example forming a liquid reaction medium (e.g., solution and/or slurry) comprising Mo, V, Nb or Ta, and Sb (as well as Ge in respect of the seventh aspect of the invention) components in the reaction vessel. Preferably, in each case, the liquid reaction medium is formed by a protocol that comprises combining components in a reaction vessel in relative molar amounts such that the aforementioned stoichiometries are met. Also preferably, in each case, the liquid reaction medium is formed by a protocol that comprises stirring while combining at least two of the components in the reaction vessel, and preferably, stirring while combining each of the components with each other in the reaction vessel. The liquid reaction media preferably comprises an aqueous solution and/or solid particulates dispersed in an aqueous carrier media. Some components, such as Mo-containing compounds and V-containing compounds and Nb-containing or Ta-containing compounds can be provided to the reaction vessel as aqueous solutions of the Mo-, V-, Nb- or Ta-, Sb-metal salts. Some of these components, as well as other components, such as Mo-containing, V-containing, Sb-containing and Ge-containing compounds can be provided to the reaction vessels as solids or as slurries comprising solid particulates dispersed in an aqueous carrier media.

Preferred precursor compounds for synthesis of the catalysts as described herein include the following. Preferred molybdenum sources include molybdenum(VI) oxide, ammonium heptamolybdate and molybdic acid. Preferred vanadium sources include vanadyl sulfate, ammonium metavanadate and vanadium(V) oxide. Preferred antimony sources include antimony(III) oxide, antimony(III) acetate, antimony(III) oxalate, antimony(V) oxide, antimony(III) sulfate, and antimony(III) tartrate. Preferred niobium sources include niobium oxalate, ammonium niobium oxalate and niobium ethoxide. Preferred tantalum sources include tantalum oxalate, ammonium tantalum oxalate, and tantalum ethoxide. A preferred germanium source is germanium(IV) oxide.

Solvents which may be used to prepare mixed metal oxides according to the invention include, but are not limited to, water, alcohols such as methanol, ethanol, propanol, diols (e.g. ethylene glycol, propylene glycol, etc.), as well as other polar solvents known in the art. Preferably, the metal precursors are soluble in the solvent, at least at the reaction temperature and pressure. Generally, water is the preferred solvent. Any water suitable for use in chemical synthesis may be used. The water may, but need not be, distilled and/or deionized.

The amount of aqueous solvent in the reaction medium may vary due to the solubilities of the precursor compounds combined to form the particular mixed metal oxide. The amount of aqueous solvent should at least be sufficient to form a slurry of the reactants. It is typical in hydrothermal synthesis of mixed metal oxides to leave an amount of headspace in the reactor vessel.

In some hydrothermal synthesis methods an oxidant may be added to the reaction medium to oxidize one or more of the metal precursors prior to the reaction step. For example, in the hydrothermal preparation of a MoVNbSb metal oxide or MoVTaSb metal oxide according to the invention, some of the V and Sb may be oxidized with an oxidant prior to the reaction step. In that case oxidant, such as H₂O₂, is added to the reaction medium. This is preferably done prior to addition of the Nb or Ta precursor compound, niobium oxalate or tantalum oxalate, to avoid unwanted reaction of the H₂O₂ with oxalic acid win the niobium or tantalum oxalate solution. Thus, when an oxidant is added to the reaction medium the order of addition may be chosen to achieve the desired oxidation and/or to avoid undesired reactions. The oxidant is preferably a non-metal-containing oxide such as H₂O₂. Metal-containing or inorganic oxidants may be used when it is desirable to introduce the particular metals or elements of the oxidant into the mixed metal oxide.

The steps of the preparation method can also comprise sealing the reaction vessel, preferably after the reaction components have been added thereto. As discussed above, it is generally desirable to maintain some headspace in the reactor vessel. The amount of headspace may depend on the vessel design or the type of agitation used if the reaction mixture is stirred. Overhead stirred reaction vessels, for example, may take 50% headspace. Typically, the headspace is filled with ambient air which provides some amount of oxygen to the reaction. However, the headspace, as is known the art, may be filled with other gases to provide reactants like O₂ or even an inert atmosphere such as Ar or N₂, the amount of headspace and gas within it depends upon the desired reaction as is known in the art.

As a further step of the preferred hydrothermal synthesis method, as generally described herein, the components are reacted in the sealed reaction vessel at a temperature greater than 100° C. and at a pressure greater than ambient pressure to form a mixed metal oxide precursor. Preferably, the components are reacted in the sealed reaction vessel at a temperature of at least about 125° C., and at a pressure of at least about 25 psig, more preferably at a temperature of at least about 150° C. and at a pressure of at least about 50 psig, and in some embodiments, at a temperature of at least about 175° C. and at a pressure of at least about 100 psig.

In any case, the components are preferably reacted by a protocol that comprises mixing the components in the sealed reaction vessel during the reaction step. The particular mixing mechanism is not narrowly critical, and can include for example, mixing (e.g., stirring or agitating) the components in the sealed reaction vessel during the reaction by any effective method. Such methods including, for example, agitating the contents of the reaction vessel, for example by shaking, tumbling or oscillating the component-containing reaction vessel. Such methods also include, for example, stirring by using a stirring member located at least partially within the reaction vessel and a driving force coupled to the stirring member or to the reaction vessel to provide relative motion between the stirring member and the reaction vessel. The stirring member can be a shaft-driven and/or shaft-supported stirring member. The driving force can be directly coupled to the stirring member or can be indirectly coupled to the stirring member (e.g., via magnetic coupling). The mixing is generally preferably sufficient to mix the components to allow for efficient reaction between components of the reaction medium to form a more homogeneous reaction medium (e.g., and resulting in a more homogeneous mixed metal oxide precursor) as compared to an unmixed reaction. Without being bound by theory not expressly recited in the claims, the well-mixed (e.g., well-stirred) reaction medium can in some cases result in a mixed metal oxide precursor, or upon further processing a mixed metal oxide catalyst, and in either case, where at least a portion of the precursor or catalyst comprises a substantially homogeneous mixture of the required elements as discussed above (e.g., as a single phase), and for example in some cases, as solid state solution, and further in some of such cases, where at least a portion thereof has the requisite crystalline structure for active and selective propane oxidation and/or ammoxidation catalysts.

Also preferably, the components can be reacted in the sealed reaction vessel at a initial pH of not more than about 4. Over the course of the hydrothermal synthesis, the pH of the reaction mixture may change such that the final pH of the reaction mixture may be higher or lower than the initial pH. Preferably, the components are reacted in the sealed reaction vessel at a pH of not more than about 3.5. In some embodiments, the components can be reacted in the sealed reaction vessel at a pH of not more than about 3.0, of not more than about 2.5, of not more than about 2.0, of not more than about 1.5 or of not more than about 1.0, of not more than about 0.5 or of not more than about 0. Preferred pH ranges include a pH ranging from about −0.5 to about 4, preferably from about 0 to about 4, more preferably from about 0.5 to about 3.5. In some embodiments, the pH can range from about 0.7 to about 3.3, or from about 1 to about 3. The pH may be adjusted by adding acid or base to the reaction mixture.

The components can be reacted in the sealed reaction vessels at the aforementioned reaction conditions (including for example, reaction temperatures, reaction pressures, pH, stirring, etc., as described above) for a period of time sufficient to form the mixed metal oxide, preferably where the mixed metal oxide comprises a solid state solution comprising the required elements as discussed above, and at least a portion thereof preferably having the requisite crystalline structure for active and selective propane or isobutane oxidation and/or ammoxidation catalysts, as described below. The exact period of time is not narrowly critical, and can include for example at least about six hours, at least about twelve hours, at least about eighteen hours, at least about twenty-four hours, at least about thirty hours, at least about thirty-six hours, at least about forty-two hours, at least about forty-eight hours, at least about fifty-four hours, at least about sixty hours, at least about sixty-six hours or at least about seventy-two hours. Reaction periods of time can be even more than three days, including for example at least about four days, at least about five days, at least about six days, at least about seven days, at least about two weeks or at least about three weeks or at least about one month.

Following the reaction step, further steps of the preferred catalyst preparation methods can include work-up steps, including for example cooling the reaction medium comprising the mixed metal oxide (e.g., to about ambient temperature), separating the solid particulates comprising the mixed metal oxide from the liquid (e.g., by centrifuging and/or decanting the supernatant, or alternatively, by filtering), washing the separated solid particulates (e.g., using distilled water or deionized water), repeating the separating step and washing steps one or more times, and effecting a final separating step.

After the work-up steps, the washed and separated mixed metal oxide can be dried. Drying the mixed metal oxide can be effected under ambient conditions (e.g., at a temperature of about 25° C. at atmospheric pressure), and/or in an oven, for example, at a temperature ranging from about 40° C. to about 150° C., and preferably of about 120° C. over a drying period of about time ranging from about five to about fifteen hours, and preferably of about twelve hours. Drying can be effected under a controlled or uncontrolled atmosphere, and the drying atmosphere can be an inert gas, an oxidative gas, a reducing gas or air, and is typically and preferably air.

As a further preparation step, the dried mixed metal oxide can be treated to form the mixed metal oxide catalyst. Such treatments can include for example calcinations (e.g., including heat treatments under oxidizing or reducing conditions) effected under various treatment atmospheres. The work-up mixed metal oxide can be crushed or ground prior to such treatment, and/or intermittently during such pretreatment. Preferably, for example, the dried mixed metal oxide can be optionally crushed, and then calcined to form the mixed metal oxide catalyst. The calcination is preferably effected in an inert atmosphere such as nitrogen. Preferred calcination conditions include temperatures ranging from about 400° C. to about 700° C., more preferably from about 500° C. to about 650° C., and in some embodiments, the calcination can be at about 600 oc.

The treated (e.g., calcined) mixed metal oxide can be further mechanically treated, including for example by grinding, sieving and pressing the mixed metal oxide. Preferable, the catalyst is sieved to form particles having a particle size distribution with a mean particle size ranging from about 100 μm to about 4001m, preferably from about 120 μm to about 380 μm, and preferably from about 140 μm to about 360 μm.

Catalyst Compositions Prepared by Aforementioned Synthesis Methods

The invention is directed, in another eighth aspect, to catalyst compositions prepared according to the general preparation protocols described above, including preferably as applied in connection with of the fifth, sixth and seventh aspects of the invention as described above.

Oxidation States/Crystalline Structures

The oxidation state of the various catalysts components as described above can vary, and can include more than one oxidation state for each of the various components. The mixed metal oxide catalyst preferably comprises one or more phases having a crystalline structure that is active and selective for propane oxidation and/or ammoxidation to form acrylic acid and/or acrylonitrile, respectively, or for isobutane to form methacrylic acid and/or methacrylonitrile, respectively.

Conversion of Propane and Isobutane via Oxidation or Ammoxidation Reactions

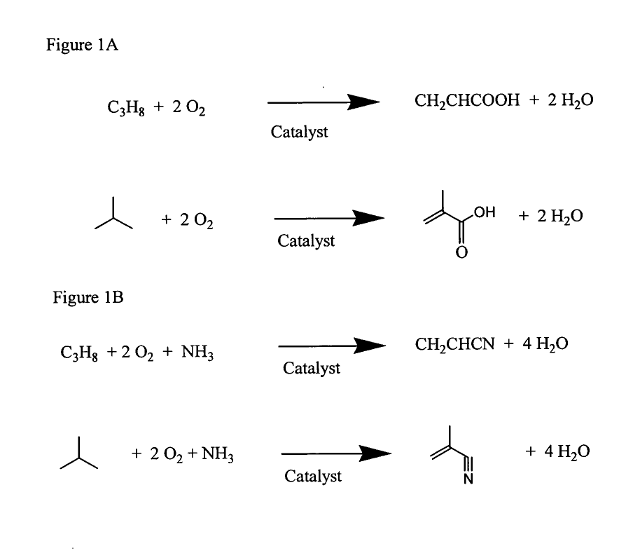

The compositions and mixed metal oxide catalysts as described in the aforementioned aspects of the invention can be used in a further ninth aspect of the invention, as a catalyst for conversion of propane to acrylic acid via an oxidation reaction or isobutane to methacrylic acid, and/or in a further tenth aspect of the invention or for conversion of propane to acrylonitrile or isobutane to methacrylonitrile via an ammoxidation reaction. FIG. 1A shows the general reaction scheme for propane oxidation to acrylic acid and isobutane to methacrylic acid, and FIG. 1B shows the general reaction scheme for propane ammoxidation to acrylonitrile and isobutane to methacrylonitrile.

Propane is preferably converted to acrylic acid and isobutane to methacrylic acid by providing one or more of the aforementioned catalysts in a gas-phase flow reactor, and contacting the catalyst with propane in the presence of oxygen (e.g. provided to the reaction zone in a feedstream comprising an oxygen-containing gas, such as and typically air) under reaction conditions effective to form acrylic acid. The feed stream for this reaction preferably comprises propane and an oxygen-containing gas such as air in a molar ratio of propane or isobutane to oxygen ranging from about 0.15 to about 5, and preferably from about 0.25 to about 2. The feed stream can also comprise one or more additional feed components, including acrylic acid or methacrylic acid product (e.g., from a recycle stream or from an earlier-stage of a multi-stage reactor), and/or steam. For example, the feedsteam can comprise about 5% to about 30% by weight relative to the total amount of the feed stream, or by mole relative to the amount of propane or isobutane in the feed stream.

Propane is preferably converted to acrylonitrile, and isobutane to methacrylonitrile, by providing one or more of the aforementioned catalysts in a gas-phase flow reactor, and contacting the catalyst with propane or isobutane in the presence of oxygen (e.g. provided to the reaction zone in a feedstream comprising an oxygen-containing gas, such as and typically air) and ammonia under reaction conditions effective to form acrylonitrile or methacrylonitrile. For this reaction, the feed stream preferably comprises propane or isobutane, an oxygen-containing gas such as air, and ammonia with the following molar ratios of: propane or isobutane to oxygen in a ratio ranging from about 0.125 to about 5, and preferably from about 0.25 to about 2.5, and propane or isobutane to ammonia in a ratio ranging from about 0.3 to about 2.5, and preferably from about 0.5 to about 1.5. The feed stream can also comprise one or more additional feed components, including acrylonitrile or methacrylonitrile product (e.g., from a recycle stream or from an earlier-stage of a multi-stage reactor), and/or steam. For example, the feedsteam can comprise about 5% to about 30% by weight relative to the total amount of the feed stream, or by mole relative to the amount of propane or isobutane in the feed stream.

For either of the above-mentioned reactions of the ninth and tenth aspects of the invention, the catalytically active mixed metal oxide composition can be provided to the reactor as a supported catalyst or as an unsupported bulk catalyst. Supports or binders for use as a supported catalyst include silica, alumina, titania, zirconia, etc. Such supported catalysts can be prepared by adding such supports (e.g., 20% to 50% by weight) to the reaction medium during the reaction step of the aforementioned preparation methods. If supported catalysts are used, the catalyst loading preferably ranges from about 50% to about 80%.

The specific design of the gas-phase flow reactor is not narrowly critical. Hence, the gas-phase flow reactor can be a fixed-bed reactor, a fluidized-bed reactor, or another type of reactor. The reactor can be a single reactor, or can be one reactor in a multi-stage reactor system. Preferably, the reactor comprises one or more feed inlets for feeding a reactant feedstream to a reaction zone of the reactor, a reaction zone comprising the mixed metal oxide catalyst, and an outlet for discharging reaction products and unreacted reactants.

The reaction conditions are controlled to be effective for converting the propane to acrylic acid or to acrylonitrile, respectively, or the isobutane to methacrylic acid or methacrylonitrile, respectively. Generally, reaction conditions include a temperature ranging from about 300° C. to about 550° C., preferably from about 325° C. to about 500° C., and in some embodiments from about 350° C. to about 450° C., and in other embodiments from about 430° C. to about 520° C. Generally, the flow rate of the propane- or isobutane-containing feedstream through the reaction zone of the gas-phase flow reactor can be controlled to provide a weight hourly space velocity (WHSV) ranging from about 0.02 to about 5, preferably from about 0.05 to about 1, and in some embodiments from about 0.1 to about 0.5, in each case, for example, in grams propane or isobutane to grams of catalyst. The pressure of the reaction zone can be controlled to range from about 0 psig to about 200 psig, preferably from about 0 psig to about 100 psig, and in some embodiments from about 0 psig to about 50 psig.

The reaction conditions can be further controlled with respect to heat transfer and/or temperature. For example, the reaction zone of the reactor is preferably configured to control heat transfer in the reaction zone, and/or temperature in the reaction zone. For example, the propane and isobutane oxidation and propane ammoxidation reactions are exothermic, and as such, the reaction zone can be cooled by one or more approaches known in the art.

Preferably, one or more of the mixed metal oxide catalyst composition, the feed compositions, and the reaction conditions are controlled to form the desired reaction product (i.e., acrylic acid and/or acrylonitrile, or methacrylic acid and/or methacrylonitrile) with a yield of at least about 50%, preferably with a yield of at least about 53% or more, and most preferably with a yield of at least about 55% or more. As used herein, the yield is calculated for the propane oxidation and/or ammoxidation reaction as described in Example 5.

The resulting acrylic acid and/or acrylonitrile or methacrylic and/or methacrylonitrile product can be isolated, if desired, from other side-products and/or from unreacted reactants according to methods known in the art.

The resulting acrylic acid and/or acrylonitrile or methacrylic acid and/or methacrylonitirle product can be used as reactant sources for numerous further (e.g., downstream) applications, according to methods known in the art.

The following examples illustrate the principles and advantages of the invention.

### EXAMPLES

### Example 1

A catalyst was prepared where the atomic ratio of Mo/V/Sb/Nb was 1/0.37/0.13/0.1 in the synthesis mixture. To a 7.0 mL Teflon lined reaction vessel was added 2 mL distilled water, (0.50 g), VOSO₄ (1.27 mL of a 1.0 M soln.), and Sb₂O₃ (0.0675 g). H₂O₂ (0.017 mL of a 30% soln.) was added to the slurry while stirring. A niobium oxalate solution was prepared by dissolving niobic acid in an oxalic acid solution at 60° C. The oxalate/Nb ratio of this solution was 3 and the concentration of Nb was 0.412 M. A portion of the niobium oxalate solution (0.841 mL of a 0.413 M soln.) was added. Distilled water was added to the reaction vessel to a 75% fill volume. The initial pH of the reaction medium was 1.2. The vessel was sealed and heated to 175° C. for 48 h without agitation. The reactor was then allowed to cool to room temperature. The solid reaction products were separated from the liquid and washed with distilled water three times. The solid was then deed in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 2

A catalyst was prepared where the atomic ratio of Mo/V/Sb/Nb/Ge was 1/0.5/0.15/0.1/0.083 in the synthesis mixture. To a 7.0 mL Teflon lined reaction vessel was added 2 mL distilled water, MoO₃ (0.50 g), VOSO₄ (1.74 mL of a 1.0 M soln.), GeO₂ (0.030 g), and Sb₂O₃ (0.076 g). H₂O₂ (0.059 mL of a 30% soln.) was added to the slurry while stirring. A niobium oxalate solution was prepared by dissolving niobic acid in an oxalic acid solution at 60° C. The oxalate/Nb ratio of this solution was 3 and the concentration of Nb was 0.413 M. A portion of the niobium oxalate solution (0.841 mL of a 0.413 M soln) was added. Distilled water was added to the reaction vessel to a 75% fill volume. The initial pH of the reaction medium was 1.2. The vessel was sealed and heated to 175° C. for 48 h without agitation. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 3

A catalyst was prepared where the atomic ratio of Mo/V/Sb/Nb was 1/0.4/0.3/0.06 in the synthesis mixture. To a 7.0 mL Teflon lined reaction vessel was added 2 mL distilled water. The water was stirred with a magnetic stir bar while adding MoO₃ (0.50 g), VOSO₄ (1.39 mL of a 1.0 M soln.), and Sb₂O₃ (0.152 g). H₂O₂ (0.106 mL of a 30% soln.) was added dropwise to the slurry and stirring was continued for 15 min. A niobium oxalate solution was prepared by dissolving niobic acid in an oxalic acid solution at 60° C. The oxalate/Nb ratio of this solution was 3 and the concentration of Nb was 0.412 M. A portion of the niobium oxalate solution (0.506 mL of a 0.412 M soln.) was added. Distilled water was added to the reaction vessel to a 75% fill volume. The initial pH of the reaction medium was 1.2. The vessel was sealed and heated to 175° C. for 48 h. During the heating the vessel was tumbled to affect agitation of the reaction medium. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 4

A catalyst was prepared where the atomic ratio of Mo/V/Sb/Nb/Ge was 1/0.3/0.3/0.06/0.8 in the synthesis mixture. To a 7.0 mL Teflon lined reaction vessel was added 2 mL distilled water. The water was stirred with a magnetic stir bar while adding MoO₃ (0.50 g), VOSO₄ (1.04 mL of a 1.0 M soln.), GeO₂ (0.291 g), and Sb₂O₃ (0.152 g). A niobium oxalate solution was prepared by dissolving niobic acid in an oxalic acid solution at 60° C. The oxalate/Nb ratio of this solution was 3 and the concentration of Nb was 0.412 M. A portion of the niobium oxalate solution (0.506 mL of a 0.412 M soln) was added. Distilled water was added to the reaction vessel to a 75% fill volume. The vessel was sealed and heated to 175° C. for 48 h. During the heating the vessel was tumbled to affect agitation of the reaction medium. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 5

The catalysts prepared as described in Examples 1 through 4 were tested for the ammoxidation of propane to acrylonitrile in a fixed bed reactor. A 150 mg sample of the catalyst was mixed with three times the volume of silicon carbide. The mixture was packed into a glass lined steel tube with a 4 mm ID. The reaction conditions were: atmospheric pressure, 420 or 430° C., WHSV=0.148 h⁻¹, feed ratio C₃H₈/NH₃/O₂/He=1/1.2/3/12. The effluent of the reactor was analyzed by gas chromatography using a Plot-Q and a molecular sieve column with FID and TCD detectors, respectively. Conversion, selectivity, and yield were defined as: Conversion=(moles C₃H₈ consumed/moles C₃H₈ charged)×100, Selectivity=(moles product/moles C₃H₈ consumed)×(# C atoms in product/3)×100, Yield=(moles product/moles C₃H₈ charged)×(# C atoms in product/3)×100. The results are shown in Table 1.

TABLE 1

| | | Reaction | AN | C₃H₈ | AN |
|---|---|---|---|---|---|
| | | Temp | Yield | Conversion | Selectivity |
| Example 1 | Mo₁V_{0.37}Nb_{0.1}Sb_{0.13}Oₓ | 420 C. | 45% | 81% | 56% |
| Example 2 | Mo₁V_{0.5}Nb_{0.1}Sb_{0.15}Ge_{0.08}Oₓ | 420 C. | 52% | 81% | 64% |
| Example 3 | Mo₁V_{0.4}Nb_{0.06}Sb_{0.3}Oₓ | 420 C. | 48% | 80% | 61% |
| Example 3 | Mo₁V_{0.4}Nb_{0.06}Sb_{0.3}Oₓ | 430 C. | 53% | 85% | 63% |
| Example 4 | Mo₁V_{0.3}Nb_{0.06}Sb_{0.3} Ge_{0.8}Oₓ | 420 C. | 54% | 82% | 66% |
| Example 4 | Mo₁V_{0.3}Nb_{0.06}Sb_{0.3} Ge_{0.8}Oₓ | 430 C. | 57% | 86% | 65% |

### Example 6

A catalyst was prepared where the ratio of Mo/V/Sb/Nb/H₂O₂ was 1/0.4/0.3/0.06/0.3 in the synthesis mixture. To a 7.0 mL Teflon lined reaction vessel was added 2 mL distilled water, MoO₃ (0.50 g), VOSO₄ (1.39 mL of a 1.0 M soln.), and Sb₂O₃ (0.152 g). H₂O₂ (0.106 mL of a 30% soln.) was added to the slurry while stirring. A niobium oxalate solution was prepared by dissolving niobic acid in an oxalic acid solution at 60° C. The oxalate/Nb ratio of this solution was 3 and the concentration of Nb was 0.42 M. A portion of the niobium oxalate solution (0.496 mL of a 0.42 M soln.) was added. Distilled water was added to the reaction vessel to a 75% fill volume. The vessel was sealed and heated to 175° C. for 48 h. During the heating the vessel was tumbled to affect agitation of the reaction medium. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 7

A catalyst was prepared by the same method as in example 6 except that H₂SO₄ (0.0191 mL of a 18.2M soln.) was added to the synthesis mixture with stirring after the H₂O₂ addition.

### Example 8

(1216_{—}9_{—}12) A catalyst was prepared by the same method as in example 6 except that H₂SO₄ (0.0954 mL of a 18.2M soln.) was added to the synthesis mixture with stirring after the H₂O₂ addition.

### Example 9

A catalyst was prepared by the same method as in example 6 except that H₂SO₄ (0.191 mL of a 18.2M soln.) was added to the synthesis mixture with stirring after the H₂O₂ addition.

### Example 10

A catalyst was prepared by the same method as in example 6 except that NH₄OH (0.233 mL of a 7.45M soln.) was added to the synthesis mixture with stirring after the H₂O₂ addition.

### Example 11

A catalyst was prepared by the same method as in example 6 except that NH₄OH (0.350 mL of a 7.45M soln.) was added to the synthesis mixture with stirring after the H₂O₂ addition.

### Example 12

A catalyst was prepared where the ratio of Mo/V/Sb/Nb/H₂O₂ was 1/0.4/0.3/0.06/0.3 in the synthesis mixture. To a 7.0 mL Teflon lined reaction vessel was added 2 mL distilled water, MoO₃ (0.50 g), NH₄VO₃ (0.163 g), and Sb₂O₃ (0.152 g). H₂O₂ (0.106 mL of a 30% soln.) was added to the slurry while stirring. A niobium oxalate solution was prepared by dissolving niobic acid in an oxalic acid solution at 60° C. The oxalate/Nb ratio of this solution was 3 and the concentration of Nb was 0.42 M. A portion of the niobium oxalate solution (0.496 mL of a 0.42 M soln.) was added. Distilled water was added to the reaction vessel to a 75% fill volume. The vessel was sealed and heated to 175° C. for 48 h. During the heating the vessel was tumbled to affect agitation of the reaction medium. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 13

A catalyst was prepared by the same method as in example 12 except that H₂SO₄ (0.0382 mL of a 18.2M soln.) was added to the synthesis mixture with stirring after the H₂O₂ addition.

### Example 14

(1216_{—}9_{—}34) A catalyst was prepared by the same method as in example 12 except that H₂SO₄ (0.0573 mL of a 18.2M soln.) was added to the synthesis mixture with stirring after the H₂O₂ addition.

### Example 15

A catalyst was prepared by the same method as in example 12 except that H₂SO₄ (0.0763 mL of a 18.2M soln.) was added to the synthesis mixture with stirring after the H₂O₂ addition.

### Example 16

A catalyst was prepared by the same method as in example 12 except that H₂SO₄ (0.0954 mL of a 18.2M soln.) was added to the synthesis mixture with stirring after the H₂O₂ addition.

### Example 17

A catalyst was prepared where the ratio of Mo/V/Sb/Nb/H₂O₂ was 1/0.4/0.3/0.06/0.3 in the synthesis mixture. To a 7.0 mL Teflon lined reaction vessel was added 2 mL distilled water, ammonium heptamolybdate (0.50 g), NH₄VO₃ (0.133 g), and Sb₂O₃ (0.124 g). H₂O₂ (0.0868 mL of a 30% soln.) was added to the slurry while stirring. A niobium oxalate solution was prepared by dissolving niobic acid in an oxalic acid solution at 60° C. The oxalate/Nb ratio of this solution was 3 and the concentration of Nb was 0.42 M. A portion of the niobium oxalate solution (0.405 mL of a 0.42 M soln.) was added. Distilled water was added to the reaction vessel to a 75% fill volume. The vessel was sealed and heated to 175° C. for 48 h. During the heating the vessel was tumbled to affect agitation of the reaction medium. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 18

A catalyst was prepared where the ratio of Mo/V/Sb/Nb/H₂O₂ was 1/0.4/0.3/0.06/0.3 in the synthesis mixture. To a 7.0 mL Teflon lined reaction vessel was added 2 mL distilled water, ammonium heptamolybdate (0.50 g), VOSO₄ (1.133 mL of a 1.0 M soln.), and Sb₂O₃ (0.124 g). H₂O₂ (0.0868 mL of a 30% soln.) was added to the slurry while stirring. A niobium oxalate solution was prepared by dissolving niobic acid in an oxalic acid solution at 60° C. The oxalate/Nb ratio of this solution was 3 and the concentration of Nb was 0.42 M. A portion of the niobium oxalate solution (0.405 mL of a 0.42 M soln.) was added. Distilled water was added to the reaction vessel to a 75% fill volume. The vessel was sealed and heated to 175° C. for 48 h. During the heating the vessel was tumbled to affect agitation of the reaction medium. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 19

During the synthesis of the samples in examples 6 through 18 the pH of the reaction medium was measured immediately prior to sealing the pressure vessel for hydrothermal synthesis and after the vessel was opened after the hydrothermal synthesis. The conductivity of the supernatant liquid of the reaction medium was measured after the hydrothermal treatment. The conductivity is reported in milisiemens. The results are shown in table 2.

TABLE 2

| | | | | | | | | | | | Final |
|---|---|---|---|---|---|---|---|---|---|---|---|
| | | | | Mo | Reaction | AN | C₃H₈ | AN | Init. | Final | Conductivity |
| | H₂SO₄^{a} | NH₄OH^{a} | V Source | Source | Temp | Yield | Conversion | Selectivity | pH^{b} | pH^{c} | (mS) |
| Example 6 | 0 | 0 | VOSO4 | MoO3 | 420 | 47.5 | 81.1 | 58.5 | 1.2 | 1.4 | 12.65 |
| Example 6 | 0 | 0 | VOSO4 | MoO3 | 430 | 48.3 | 85.2 | 56.7 | 1.2 | 1.4 | 12.65 |
| Example 7 | 0.1 | 0 | VOSO4 | MoO3 | 420 | 3.4 | 12.7 | 26.3 | 1 | 1.4 | 17.6 |
| Example 8 | 0.5 | 0 | VOSO4 | MoO3 | 420 | 0.2 | 1.0 | 16.7 | 1 | 1 | 23.6 |
| Example 9 | 1 | 0 | VOSO4 | MoO3 | 420 | 0.1 | 0.2 | 34.8 | 0.8 | 1 | 29.8 |
| Example 10 | 0 | 0.5 | VOSO4 | MoO3 | 420 | 31.2 | 81.0 | 38.6 | 2.8 | 2.3 | 7.11 |
| Example 11 | 0 | 0.75 | VOSO4 | MoO3 | 420 | 29.2 | 80.1 | 36.4 | 2.8 | 2.5 | 7.29 |
| Example 12 | 0 | 0 | NH4VO3 | MoO3 | 420 | 1.2 | 13.1 | 9.2 | 2.8 | 5.1 | 0.558 |
| Example 13 | 0.2 | 0 | NH4VO3 | MoO3 | 420 | 45.4 | 84.6 | 53.7 | 1.8 | 2.3 | 5.05 |
| Example 14 | 0.3 | 0 | NH4VO3 | MoO3 | 420 | 49.6 | 88.8 | 55.8 | 1.2 | 2 | 5.25 |
| Example 15 | 0.4 | 0 | NH4VO3 | MoO3 | 420 | 45.6 | 87.5 | 52.1 | 1 | 1.8 | 7.34 |
| Example 16 | 0.5 | 0 | NH4VO3 | MoO3 | 420 | 46.3 | 84.0 | 55.1 | 1 | 1.6 | 9.51 |
| Example 17 | 0 | 0 | NH4VO3 | Mo7O24 | 420 | 0.8 | 6.3 | 12.7 | 2.3 | 4.4 | 0.086 |
| Example 18 | 0 | 0 | VOSO4 | Mo7O24 | 420 | 11.3 | 19.4 | 58.0 | 1 | 1.4 | 11.17 |

^{a}Molar ratio relative to Mo.
^{b}Initial pH immediately prior to hydrothermal treatment of the reaction medium.
^{c}Final pH of the reaction medium after hydrothermal treatment.

Comparative Examples 20-24 illustrate MoVTeNbOₓ catalyst prepared by solvent evaporation (SE) with and without oxalic acid and calcined under various conditions. As shown in Table 3 below, when oxalic acid is added to the synthesis mixture and the material is calcined at 600° C. under N₂ the catalyst is poor. If the material with added oxalic acid is calcined in air at 280° C. and then under N₂ at 600° C. the performance of the catalyst is similar to the one prepared without oxalic acid. Thus, for the remaining Examples done with added oxalic acid or Ge oxalate, the materials were calcined in air at 280° C. and then under N₂ at 600° C.

### Comparative Example 20

A catalyst was prepared where the atomic ratio of Mo/V/Te/Nb was 1/0.32/0.2/0.1 in the synthesis mixture. To a 100 mL flask was added 25 mL distilled water, (NH₄)₆Mo₇O₂₄ (1.412 g) and NH₄VO₃ (0.299 g). The mixture was heated to 70° C. until the solids dissolved. The solution was cooled to room temperature and Te(OH)₆ (0.367 g) was added and allowed to dissolve. A niobium oxalate solution was prepared by dissolving niobic acid in an oxalic acid solution at 60° C. The oxalate/Nb ratio of this solution was 3 and the concentration of Nb was 0.458 M. A portion of the niobium oxalate solution (1.747 mL of a 0.458 M soln.) was added. The solvent was removed from the mixture under reduced pressure at 50° C. The solid was then dried in air at 120° C. for 12 h, crushed, and calcined under N₂ at 600° C. for 2 h. The material was ground to a fine powder in a ball mill, pressed into a pellet, crushed and sieved to 145 to 355 μm particles.

### Comparative Example 21

A catalyst was prepared with a similar method to example 1 where the atomic ratio of Mo/V/Te/Nb was 1/0.32/0.2/0.1 in the synthesis mixture. Prior to the addition of the niobium oxalate solution an oxalic acid solution (9.6 mL of a 0.5M solution) was added the MoVTe mixture. The solvent was removed from the mixture under reduced pressure at 50° C. The solid was then dried in air at 120° C. for 12 h, crushed, and calcined under N₂ at 600° C. for 2 h. The material was ground to a fine powder in a ball mill, pressed into a pellet, crushed and sieved to 145 to 355 μm particles.

Comparative Example 22. (1037_{—}91A_{—}5) A portion of the material from example 1 that was dried in air at 120° C. was further heated in air at 280° C. for 2 h. The solid was then calcined under N₂ at 600° C. for 2 h. The material was ground to a fine powder in a ball mill, pressed into a pellet, crushed and sieved to 145 to 355 μm particles.

Comparative Example 23. (1037_{—}91A_{—}6) A portion of the material from example 2 that was dried in air at 120° C. was further heated in air at 280° C. for 2 h. The solid was then calcined under N₂ at 600° C. for 2 h. The material was ground to a fine powder in a ball mill, pressed into a pellet, crushed and sieved to 145 to 355 μm particles.

### Comparative Example 24

The catalysts prepared as described in Examples 1 through 4 were tested for the ammoxidation of propane to acrylonitrile in a fixed bed reactor. A 150 mg sample of the catalyst was mixed with three times the volume of silicon carbide. The mixture was packed into a glass lined steel tube with a 4 mm ID. The reaction conditions were: atmospheric pressure, 420° C., WHSV=0.15 h⁻¹, feed ratio C₃H₈/NH₃/02/He=1/1.2/3/12. The effluent of the reactor was analyzed by gas chromatography using a Plot-Q and a molecular sieve column with FID and TCD detectors, respectively. Conversion, selectivity, and yield were defined as: Conversion=(moles C₃H₈ consumed/moles C₃H₈ charged)×100, Selectivity=(moles product/moles C₃H₈ consumed)×(# C atoms in product/3)×100, Yield=(moles product/moles C₃H₈ charged)×(# C atoms in product/3)×100. The results are shown in Table 3.

TABLE 3

| Example | | AN | C₃H₈ | AN |
|---|---|---|---|---|
| No. | | Yield | Conversion | Selectivity |
| C-20 | Mo₁V_{0.32}Te_{0.2}Nb_{0.1}Oₓ | 54% | 88% | 62% |
| C-21 | Mo₁V_{0.32}Te_{0.2}Nb_{0.1}Oₓ + | 4% | 6% | 60% |
| | oxalate_{0.6} | | | |
| C-22 | Mo₁V_{0.32}Te_{0.2}Nb_{0.1}Oₓ | 53% | 93% | 57% |
| C-23 | Mo₁V_{0.32}Te_{0.2}Nb_{0.1}Oₓ + | 39% | 58% | 67% |
| | oxalate_{0.6} | | | |

Comparative Examples 25-29 illustrate MoVTeNbOₓ+Ge which was added as Ge oxalate and MoVTeNbOₓ+oxalic acid, prepared by solvent evaporation. As shown in Table 4 below, addition of Ge lowers the performance of the catalyst, however, addition of oxalic acid does not lower the performance of the catalyst as drastically. Thus, Ge is responsible for the decrease in performance rather than the oxalate that is associated with the Ge precursor.

### Comparative Example 25

A catalyst was prepared where the atomic ratio of Mo/V/Te/Nb was 1/0.32/0.23/0.1 in the synthesis mixture. To a 50 mL flask was added 12 mL distilled water, (NH₄)₆Mo₇O₂₄ (0.500 g) and NH₄VO₃ (0.106 g). The mixture was heated to 70° C. until the solids dissolved. The solution was cooled to room temperature and Te(OH)₆ (1.303 mL of a 0.5M solution) was added. A niobium oxalate solution was prepared by dissolving niobic acid in an oxalic acid solution at 60° C. The oxalate/Nb ratio of this solution was 3 and the concentration of Nb was 0.458 M. A portion of the niobium oxalate solution (0.618 mL of a 0.458 M soln.) was added. The solvent was removed from the mixture under reduced pressure at 50° C. The solid was then dried in air at 120° C. for 12 h, then heated to 280° C. in air for 2 h, crushed, and calcined under N₂ at 600° C. for 2 h. The material was ground to a fine powder in a ball mill, pressed into a pellet, crushed and sieved to 145 to 355 μm particles.

### Comparative Example 26

A catalyst was prepared where the atomic ratio of Mo/V/Te/Nb/Ge was 1/0.32/0.23/0.1/0.1 in the synthesis mixture. To a 50 mL flask was added 12 mL distilled water, (NH₄)₆MO₇O₂₄ (0.500 g) and NH₄VO₃ (0.106 g). The mixture was heated to 70° C. until the solids dissolved. The solution was cooled to room temperature and Te(OH)₆ (1.303 mL of a 0.5M solution) was added. A germanium oxalate solution was prepared by dissolving amorphous germanium oxide in an oxalic acid solution at 60° C. The oxalate/Ge ratio of this solution was 3 and the concentration of Ge was 0.5 M. A portion of the germanium oxalate solution (0.566 mL of a 0.5 M soln.) was added. A niobium oxalate solution was prepared by dissolving niobic acid in an oxalic acid solution at 60° C. The oxalate/Nb ratio of this solution was 3 and the concentration of Nb was 0.458 M. A portion of the niobium oxalate solution (0.618 mL of a 0.458 M soln.) was added. The solvent was removed from the mixture under reduced pressure at 50° C. The solid was then dried in air at 120° C. for 12 h, then heated to 280° C. in air for 2 h, crushed, and calcined under N₂ at 600° C. for 2 h. The material was ground to a fine powder in a ball mill, pressed into a pellet, crushed and sieved to 145 to 355 μm particles.

### Comparative Example 27

A catalyst was prepared in a similar manner to Comparative Example 26 where the atomic ratio of Mo/V/Te/Nb/Ge was 1/0.32/0.23/0.1/0.3 in the synthesis mixture. The amount of germanium oxalate solution used was 1.700 mL of a 0.5 M soln.

### Comparative Example 28

A catalyst was prepared in a similar manner to Comparative Example 26 where the atomic ratio of Mo/V/Te/Nb was 1/0.32/0.23/0.1 in the synthesis mixture. Prior to the addition of the niobium oxalate solution an oxalic acid solution (1.700 mL of a 0.5M solution) was added the MoVTe mixture.

### Comparative Example 29

A catalyst was prepared in a similar manner to Comparative Example 26 where the atomic ratio of Mo/V/Te/Nb was 1/0.32/0.23/0.1 in the synthesis mixture. Prior to the addition of the niobium oxalate solution an oxalic acid solution (5.098 mL of a 0.5M solution) was added the MoVTe mixture.

TABLE 4

| Ex- | | | | |
|---|---|---|---|---|
| ample | | AN | C₃H₈ | AN |
| No. | | Yield | Conversion | Selectivity |
| C-25 | Mo₁V_{0.32}Te_{0.23}Nb_{0.1}Oₓ | 48% | 90% | 53% |
| C-26 | Mo₁V_{0.32}Te_{0.23}Nb_{0.1}Ge_{0.1}Oₓ | 16% | 41% | 38% |
| C-27 | Mo₁V_{0.32}Te_{0.23}Nb_{0.1}Ge_{0.3}Oₓ | 20% | 47% | 43% |
| C-28 | Mo₁V_{0.32}Te_{0.23}Nb_{0.1}Oₓ + | 27% | 68% | 40% |
| | oxalate_{0.1} | | | |
| C-29 | Mo₁V_{0.32}Te_{0.23}Nb_{0.1}Oₓ + | 42% | 82% | 51% |
| | oxalate_{0.9} | | | |

Comparative Examples 30-33 illustrate MoVTeNbOₓ+Ge prepared by hydrothermal synthesis (HS) using V₂O₅ as the V source. The performances of these catalysts are generally higher than the ones prepared with VOSO₄ as the V source. As shown in Table 5, for all V, Nb, and Te levels tried the Ge free analog always has a higher catalytic performance than the samples containing Ge_{0.2}.

### Comparative Example 30

A catalyst was prepared where the atomic ratio of Mo/V/Te/Nb was 1/0.36/0.2/0.06 in the synthesis mixture. To a 7.0 mL Teflon lined reaction vessel was added 2 mL distilled water, MoO₃ (0.50 g), V₂O₅ (0.1137 g), and TeO₂ (0.111 g). A niobium oxalate solution was prepared by dissolving niobic acid in an oxalic acid solution at 60° C. The oxalate/Nb ratio of this solution was 3 and the concentration of Nb was 0.458 M. A portion of the niobium oxalate solution (0.455 mL of a 0.458 M soln) was added. Distilled water was added to the reaction vessel to an 80% fill volume. The vessel was sealed and heated to 175° C. for 48 h with agitation. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Comparative Example 31

A catalyst was prepared where the atomic ratio of Mo/V/Te/Nb/Ge was 1/0.36/0.2/0.06/0.2 in the synthesis mixture. The procedure was the same as described in Comparative Example 30 except that GeO₂ (0.0727 g) was added to the synthesis slurry following the TeO₂ addition.

### Comparative Example 32

A catalyst was prepared where the atomic ratio of Mo/V/Te/Nb was 1/0.36/0.23/0.06 in the synthesis mixture. The procedure was the same as described in Comparative Example 30.

### Comparative Example 33

A catalyst was prepared where the atomic ratio of Mo/V/Te/Nb/Ge was 1/0.36/0.23/0.06/0.2 in the synthesis mixture. The procedure was the same as described in Comparative Example 30 except that GeO₂ (0.0727 g) was added to the synthesis slurry following the TeO₂ addition. The amount of TeO₂ used was 0.1275 g.

TABLE 5

| Ex- | | | | |
|---|---|---|---|---|
| ample | | AN | C₃H₈ | AN |
| No. | | Yield | Conversion | Selectivity |
| C-30 | Mo₁V_{0.36}Te_{0.2}Nb_{0.06}Oₓ | 26% | 69% | 38% |
| C-31 | Mo₁V_{0.36}Te_{0.2}Nb_{0.06}Ge_{0.2}Oₓ | 21% | 52% | 41% |
| C-32 | Mo₁V_{0.36}Te_{0.23}Nb_{0.06}Oₓ | 20% | 64% | 31% |
| C-33 | Mo₁V_{0.36}Te_{0.23}Nb_{0.06}Ge_{0.2}Oₓ | 12% | 34% | 36% |

Comparative Examples 34-40 illustrate MoVTeNbOₓ+Ge (6 levels) prepared by hydrothermal synthesis (HS) using V₂O₅ as the V source. As shown in Table 6, addition of Ge tends to lower conversion and increase selectivity. The net result is similar yields for all Ge levels when the samples are compared under the same reaction conditions.

### Comparative Example 34

A catalyst was prepared where the atomic ratio of Mo/V/Te/Nb was 1/0.36/0.2/0.06 in the synthesis mixture. To a 7.0 mL Teflon lined reaction vessel was added 2 mL distilled water, MoO₃ (0.50 g), V₂O₅ (0.114 g), and TeO₂ (0.111 g). A niobium oxalate solution was prepared by dissolving niobic acid in an oxalic acid solution at 60° C. The oxalate/Nb ratio of this solution was 3 and the concentration of Nb was 0.399 M. A portion of the niobium oxalate solution (0.522 mL of a 0.399 M soln) was added with stirring. Distilled water was added to the reaction vessel to an 80% fill volume. The vessel was sealed and heated to 175° C. for 48 h with agitation. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Comparative Example 35

A catalyst was prepared where the atomic ratio of Mo/V/Te/Nb/Ge was 1/0.36/0.2/0.06/0.05 in the synthesis mixture. The procedure was the same as described in Comparative Example 34 except that GeO₂ (0.0182 g) was added to the synthesis slurry following the TeO₂ addition.

### Comparative Example 36

A catalyst was prepared where the atomic ratio of Mo/V/Te/Nb/Ge was 1/0.36/0.2/0.06/0.1 in the synthesis mixture. The procedure was the same as described in Comparative Example 34 except that GeO₂ (0.0363 g) was added to the synthesis slurry following the TeO₂ addition.

### Comparative Example 37

A catalyst was prepared where the atomic ratio of Mo/V/Te/Nb/Ge was 1/0.36/0.2/0.06/0.15 in the synthesis mixture. The procedure was the same as described in Comparative Example 34 except that GeO₂ (0.0545 g) was added to the synthesis slurry following the TeO₂ addition.

### Comparative Example 38

A catalyst was prepared where the atomic ratio of Mo/V/Te/Nb/Ge was 1/0.36/0.2/0.06/0.2 in the synthesis mixture. The procedure was the same as described in Comparative Example 34 except that GeO₂ (0.0727 g) was added to the synthesis slurry following the TeO₂ addition.

### Comparative Example 39

A catalyst was prepared where the atomic ratio of Mo/V/Te/Nb/Ge was 1/0.36/0.2/0.06/0.3 in the synthesis mixture. The procedure was the same as described in example 15 except that GeO₂ (0.109 g) was added to the synthesis slurry following the TeO₂ addition.

### Comparative Example 40

A catalyst was prepared where the atomic ratio of Mo/V/Te/Nb/Ge was 1/0.36/0.2/0.06/0.4 in the synthesis mixture. The procedure was the same as described in Comparative Example 34 except that GeO₂ (0.145 g) was added to the synthesis slurry following the TeO₂ addition.

TABLE 6

| Ex- | | | | |
|---|---|---|---|---|
| ample | | AN | C₃H₈ | AN |
| No. | | Yield | Conversion | Selectivity |
| C-34 | Mo₁V_{0.36}Te_{0.2}Nb_{0.06}Oₓ | 24% | 72% | 34% |
| C-35 | Mo₁V_{0.36}Te_{0.2}Nb_{0.06}Ge_{0.05}Oₓ | 30% | 75% | 40% |
| C-36 | Mo₁V_{0.36}Te_{0.2}Nb_{0.06}Ge_{0.1}Oₓ | 26% | 64% | 40% |
| C-37 | Mo₁V_{0.36}Te_{0.2}Nb_{0.06}Ge_{0.15}Oₓ | 23% | 55% | 41% |
| C-38 | Mo₁V_{0.36}Te_{0.2}Nb_{0.06}Ge_{0.2}Oₓ | 25% | 58% | 42% |
| C-39 | Mo₁V_{0.36}Te_{0.2}Nb_{0.06}Ge_{0.3}Oₓ | 23% | 48% | 48% |
| C-40 | Mo₁V_{0.36}Te_{0.2}Nb_{0.06}Ge_{0.4}Oₓ | 24% | 65% | 37% |

Examples 41-46 illustrate MoVSbNbOₓ+Ge (6 levels) prepared by hydrothermal synthesis (HS) using VOSO₄ as the V source. The data shown in Table 6 generally shows (i) that Ge containing catalysts have better performance than the Ge free catalyst and (ii) that increasing the level of Ge in the catalyst does impact performance of the MoVSbNbOₓ+Ge catalysts.

### Example 41

A catalyst was prepared where the atomic ratio of Mo/V/Sb/Nb was 1/0.32/0.2/0.06 in the synthesis mixture. To a 7.0 mL Teflon lined reaction vessel was added 2 mL distilled water, MoO₃ (0.50 g), VOSO₄ (1.112 mL of a 1.0 M soln.), and Sb₂O₃ (0.1013 g). A niobium oxalate solution was prepared by dissolving niobic acid in an oxalic acid solution at 60° C. The oxalate/Nb ratio of this solution was 3 and the concentration of Nb was 0.458 M. A portion of the niobium oxalate solution (0.455 mL of a 0.458 M soln) was added to the synthesis mixture while stirring. Distilled water was added to the reaction vessel to an 80% fill volume. The vessel was sealed and heated to 175° C. for 48 h with agitation. The reactor was then allowed to cool to room temperature. The solid reaction products were separated from the liquid and washed with distilled water three times. The solid was then dried in air at 120° C. for 12 h, crushed, and calcined under N₂ at 600° C. for 2 h. The material was ground to a fine powder in a ball mill, pressed onto a pellet, crushed and sieved to 145 to 355 μm particles.

### Example 42

A catalyst was prepared where the atomic ratio of Mo/V/Te/Nb/Ge was 1/0.32/0.2/0.06/0.05 in the synthesis mixture. The procedure was the same as described in Example 41 except that GeO₂ (0.0182 g) was added to the synthesis slurry following the Sb₂O₃ addition.

### Example 43

A catalyst was prepared where the atomic ratio of Mo/V/Te/Nb/Ge was 1/0.32/0.2/0.06/0.1 in the synthesis mixture. The procedure was the same as described in Example 41 except that GeO₂ (0.0363 g) was added to the synthesis slurry following the Sb₂O₃ addition.

### Example 44

A catalyst was prepared where the atomic ratio of Mo/V/Te/Nb/Ge was 1/0.32/0.2/0.06/0.15 in the synthesis mixture. The procedure was the same as described in Example 41 except that GeO₂ (0.0545 g) was added to the synthesis slurry following the Sb₂O₃ addition.

### Example 45

A catalyst was prepared where the atomic ratio of Mo/V/Te/Nb/Ge was 1/0.32/0.2/0.06/0.2 in the synthesis mixture. The procedure was the same as described in Example 41 except that GeO₂ (0.0727 g) was added to the synthesis slurry following the Sb₂O₃ addition.

### Example 46

A catalyst was prepared where the atomic ratio of Mo/V/Te/Nb/Ge was 1/0.32/0.2/0.06/0.4 in the synthesis mixture. The procedure was the same as described in Example 41 except that GeO₂ (0.145 g) was added to the synthesis slurry following the Sb₂O₃ addition.

TABLE 7

| | | | C3H8 | |
|---|---|---|---|---|
| Example | | AN | Con- | AN |
| No. | | Yield | version | Selectivity |
| Example 41 | Mo₁V_{0.32}Sb_{0.2}Nb_{0.06}Oₓ | 41 | 77 | 53 |
| Example 42 | Mo₁V_{0.32}Sb_{0.2}Nb_{0.06}Ge_{0.05}Oₓ | 43 | 79 | 55 |
| Example 43 | Mo₁V_{0.32}Sb_{0.2}Nb_{0.06}Ge_{0.1}Oₓ | 45 | 84 | 54 |
| Example 44 | Mo₁V_{0.32}Sb_{0.2}Nb_{0.06}Ge_{0.15}Oₓ | 44 | 84 | 53 |
| Example 45 | Mo₁V_{0.32}Sb_{0.2}Nb_{0.06}Ge_{0.2}Oₓ | 41 | 82 | 50 |
| Example 46 | Mo₁V_{0.32}Sb_{0.2}Nb_{0.06}Ge_{0.4}Oₓ | 41 | 72 | 57 |

Comparative Example 47 and Examples 48-50 illustrate the conversion of propane to acrylonitrile using MoVSbNbOₓ+Ge catalyst prepared by hydrothermal synthesis (HS) various batch sizes (23 ml, 450 ml and 1 gallon).

TABLE 8

| Wwh 0.1 | C₃H₈ % Conv | Sel AN | Aceto | HCN | C₃^{═} | CO | CO₂ |
|---|---|---|---|---|---|---|---|
| Mo₁V_{0.3}Nb_{0.06}Sb_{0.20} | | | | | | | |
| Comp. Ex. 47 - 23 ml | 66 | 56 | 4 | 11 | 3 | 13 | 12 |
| Mo₁V_{0.3}Nb_{0.06}Sb_{0.20}Ge_{0.30} | | | | | | | |
| Ex. 48 - 1 gal | 82 | 48 | 3 | 14 | 1 | 14 | 19 |
| Ex. 49 - 450 ml | 82 | 54 | 4 | 11 | 1 | 15 | 14 |
| Ex. 50 - 23 ml | 86 | 52 | 3 | 14 | 1 | 14 | 16 |

The catalyst was prepared hydrothermally with the nominal composition of Mo₁V_{0.3}Nb_{0.06}Sb_{0.20}Ge_{0.30} as follows. Two solutions were initially prepared separately. The first solution contained 0.9 g VOSO₄, 0.2 grams of MoO₃, 0.41 grams of Sb₂O₃ and 0.44 grams of amorphous GeO₂. The second solution contained 0.32 grams of oxalic acid dihydrate and 0.14 grams of niobic acid heated to 60° C. The second solution was added to the first solution and the resulting mixture was placed into a Teflon lined 23 ml Paar bomb. The bomb was sealed and heated to 175° C. for 48 hours while rotating. After 48 hours, the reactor was cooled to room temperature, opened and the solids filtered, washed, dried in air at 90° C., crushed and calcined under nitrogen at 600° C. for two hours. The calcined material was pulverized to a fine powder, pressed into a pellet, crushed and sieved to the appropriate particle size. This procedure was repeated for 450 ml (Example 49) and 1-gallon (Example 48) Parr bomb reactors. This procedure was also repeated for a Ge free catalyst (Comparative Example 47).

Typically, 0.5 grams of catalyst and 2.5 grams of inert quartz chips were loaded into a small test reactor for testing. The composition of the feed gas was as follows. 1.0 C₃/1.2 NH₃/3 O₂/12 N₂. Reactor temperature was 410° C. The results of testing these catalysts for the ammoxidation of propane are shown in Table 8.

In light of the detailed description of the invention and the examples presented above, it can be appreciated that the several objects of the invention are achieved.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the examples and the embodiments of the present invention as set forth above are not intended as being exhaustive or limiting of the invention.

1. A mixed metal oxide comprising molybdenum, vanadium, niobium, antimony, germanium, and oxygen or molybdenum, vanadium, tantalum, antimony, germanium, and oxygen.

2. The mixed metal oxide of claim 1 having an essential absence of tellurium.

3. The mixed metal oxide of claim 1 having an essential absence of cerium.

4. The mixed metal oxide of claim 1 having an essential absence of gallium.

5. The mixed metal oxide of claim 1 having an essential absence of tellurium, cerium and gallium.

6. The mixed metal oxide of claim 1 consisting essentially of molybdenum, vanadium, niobium, antimony, germanium, and oxygen or molybdenum, vanadium, tantalum, antimony, germanium, and oxygen.

7. The mixed metal oxide of claim 1 wherein the stoichiometric ratios of elements include a ratio of molybdenum to germanium ranging from 1: >0.1 to about 1:1.

8. The mixed metal oxide of claim 1 wherein the stoichiometric ratios of the elements includes
a ratio of molybdenum to antimony ranging from about 1:0.1 to about 1:0.5, and
a ratio of molybdenum to germanium ranging from about 1:0.01 to about 1:1.

9. The mixed metal oxide of claim 1 wherein the stoichiometric ratios of the elements includes
a ratio of molybdenum to vanadium ranging from about 1:0.1 to about 1:0.6,
a ratio of molybdenum to niobium or tantalum ranging from about 1:0.02 to about 1:0.12,
a ratio of molybdenum to antimony ranging from about 1:0.1 to about 1:0.5, and
a ratio of molybdenum to germanium ranging from about 1:0.01 to about 1:1.

10. A catalyst comprising a mixed metal oxide effective for vapor phase conversion of propane to acrylic acid or to acrylonitrile or of isobutane to methacrylic acid or to methacrylonitrile, the mixed metal oxide comprising molybdenum, vanadium, niobium, antimony, germanium, and oxygen or molybdenum, vanadium, tantalum, antimony, germanium, and oxygen.

11. The catalyst of claim 10 wherein the mixed metal oxide has an essential absence of tellurium.

12. The catalyst of claim 10 wherein the mixed metal oxide has an essential absence of cerium.

13. The catalyst of claim 10 wherein the mixed metal oxide has an essential absence of gallium.

14. The catalyst of claim 10 wherein the mixed metal oxide has an essential absence of tellurium, cerium and gallium.

15. The catalyst of claim 10 wherein the mixed metal oxide composition consists essentially of molybdenum, vanadium, niobium, antimony, germanium, and oxygen or of molybdenum, vanadium, tantalum, antimony, germanium, and oxygen.

16. The catalyst of claim 10 wherein the stoichiometric ratios of the elements of the mixed metal oxide includes a ratio of molybdenum to germanium ranging from about 1: >0.1 to about 1:1.

17. The catalyst of claim 10 wherein the stoichiometric ratios of the elements of the mixed metal oxide includes
a ratio of molybdenum to antimony ranging from about 1:0.1 to about 1:0.5, and
a ratio of molybdenum to germanium ranging from about 1:0.01 to about 1:1.

18. The catalyst of claim 10 wherein the stoichiometric ratios of the elements of the mixed metal oxide includes
a ratio of molybdenum to vanadium ranging from about 1:0.1 to about 1:0.6,
a ratio of molybdenum to niobium or tantalum ranging from about 1:0.02 to about 1:0.12,
a ratio of molybdenum to antimony ranging from about 1:0.1 to about 1:0.5, and
a ratio of molybdenum to germanium ranging from about 1:0.01 to about 1:1.

19. A catalyst comprising a mixed metal oxide effective for vapor phase conversion of propane to acrylic acid or acrylonitrile of isobutane to methacrylic acid or to methacrylonitrile, the mixed metal oxide having the empirical formula
Mo₁VₐNb_{b}Sb_{c}Ge_{d}Oₓ or Mo₁VₐTa_{b}Sb_{c}Ge_{d}Oₓ
wherein
a ranges from about 0.1 to about 0.6,
b ranges from about 0.02 to about 0.12,
c ranges from about 0.1 to about 0.5,
d ranges from about 0.01 to about 1, and
x depends on the oxidation state of other elements present in the mixed metal oxide.

20. The catalyst of claim 19, wherein d ranges from greater than 0.1 to about 1.

21. The catalyst of claim 19 wherein the mixed metal oxide has an essential absence of tellurium.

22. The catalyst of claim 19 wherein the mixed metal oxide has an essential absence of cerium.

23. The catalyst of claim 19 wherein the mixed metal oxide has an essential absence of gallium.

24. The catalyst of claim 19 wherein the mixed metal oxide has an essential absence of tellurium, cerium and gallium.

25. The catalyst of claim 19 wherein the mixed metal oxide consists essentially of molybdenum, vanadium, niobium, antimony, germanium, and oxygen or of molybdenum, vanadium, tantalum, antimony, germanium, and oxygen.

26. The catalyst of claim 19 wherein the mixed metal oxide further comprises one or more additional elements.

27. The catalyst of claim 19 wherein the mixed metal oxide further comprises one or more additional elements selected from the group consisting of alkali metals, alkaline earth metals, rare earth metals, lanthanides and transition metals and main group metals.

28. The catalyst of claim 19 wherein mixed metal oxide is a supported mixed metal oxide.

29. The catalyst of claim 19 wherein the mixed metal oxide further comprises one or more binders.

30. A method for preparing a mixed metal oxide comprising molybdenum, vanadium, niobium or tantalum, and antimony comprising the steps of:
admixing, in a reaction vessel, precursor compounds of Mo, V, Nb or Ta, and Sb in an aqueous solvent to form a reaction medium having an initial pH of 4 or less;
optionally adding additional aqueous solvent to the reaction vessel;
sealing the reaction vessel;
reacting the reaction medium at a temperature greater than 100° C. and a pressure greater than ambient pressure for a time sufficient to form a mixed metal oxide;
optionally cooling the reaction medium; and
recovering the mixed metal oxide from the reaction medium.

31. A method of claim 30, wherein the admixing step occurs with agitation.

32. A method of claim 31, wherein the admixing step comprises the steps of
admixing precursor compounds of Mo, V, and Sb;
adding an oxidant to oxidize at least some of the V and Sb; and
after the V and Sb oxidation is substantially complete, adding an aqueous solution of niobium oxalate as the compound of Nb or of Ta.

33. A method of claim 32, wherein the oxidant is H₂O₂.

34. A method of claim 30, further comprising, after the recovery step, the steps of:
optionally washing the recovered mixed metal oxide;
drying the recovered mixed metal oxide; and
calcining the recovered mixed metal oxide.

35. A method of claim 30, wherein the mixed metal oxide has the empirical formula Mo₁VₐNb_{b}Sb_{c}Oₓ, and in the admixing step the compounds of Mo, V, Nb and Sb are added in relative molar amounts such that a ranges from about 0.1 to about 0.6, b ranges from about 0.02 to about 0.12, c ranges from about 0.1 to about 0.5, and x depends on the oxidation state of other elements present in the final mixed metal oxide, or the empirical formula Mo₁VₐTa_{b}Sb_{c}Oₓ, and in the admixing step the compounds of Mo, V, Ta and Sb are added in relative molar amounts such that a ranges from about 0.1 to about 0.6, b ranges from about 0.02 to about 0.12, c ranges from about 0.1 to about 0.5, and x depends on the oxidation state of other elements present in the final mixed metal oxide.

36. The method of claim 30 or of claims depending therefrom, wherein the reaction medium has a pH of not more than about 1.5.

37. A method of claim 30, wherein the mixed metal oxide further comprises germanium and the admixing step further comprises admixing a compound of Ge.

38. A method of claim 37, further comprising, after the recovery step, the steps of:
optionally washing the recovered mixed metal oxide;
drying the recovered mixed metal oxide; and
calcining the recovered mixed metal oxide.

39. A method of claim 37, wherein the mixed metal oxide has the empirical formula Mo₁VₐNb_{b}Sb_{c}Ge_{d}Oₓ, and in the admixing step the compounds of Mo, V, Nb, Sb and Ge are added in relative molar amounts such that a ranges from about 0.1 to about 0.6, b ranges from about 0.02 to about 0.12, c ranges from about 0.1 to about 0.5, d ranges from about 0.01 to about 1, and x depends on the oxidation state of other elements present in the mixed metal oxide, or the empirical formula Mo₁VₐTa_{b}Sb_{c}Ge_{d}Oₓ, and in the admixing step the compounds of Mo, V, Ta, Sb and Ge are added in relative molar amounts such that a ranges from about 0.1 to about 0.6, b ranges from about 0.02 to about 0.12, c ranges from about 0.1 to about 0.5, d ranges from about 0.01 to about 1, and x depends on the oxidation state of other elements present in the final mixed metal oxide.

40. A method of claim 39, wherein d, in both empirical formulas, ranges from greater than 0.1 to about 1.

41. A method for preparing a mixed metal oxide comprising molybdenum, vanadium, niobium or tantalum, and antimony comprising the steps of:
admixing, in a reaction vessel, precursor compounds of Mo, V, Nb or Ta, and Sb in an aqueous solvent to form a reaction medium;
optionally adding additional aqueous solvent to the reaction vessel;
sealing the reaction vessel;
reacting the reaction medium at a temperature greater than 100° C. and a pressure greater than ambient pressure while agitating the reaction medium for a time sufficient to form a mixed metal oxide;
optionally cooling the reaction medium; and
recovering the mixed metal oxide from the reaction medium.

42. A method of claim 41, wherein the admixing step occurs with agitation.

43. A method of claim 41, wherein the admixing step comprises the steps of
admixing precursor compounds of Mo, V, and Sb;
adding an oxidant to oxidize at least some of the V and Sb; and
after the V and Sb oxidation is substantially complete, adding an aqueous solution of niobium oxalate as the compound of Nb or an aqueous solution of tantalum oxalate as the compound of Ta.

44. A method of claim 43, wherein the oxidant is H₂O₂.

45. A method of claim 41, wherein the initial pH of the reaction medium is 3 or less.

46. A method of claim 41, further comprising, after the recovery step, the steps of:
optionally washing the recovered mixed metal oxide;
drying the recovered missed metal oxide; and
calcining the recovered mixed metal oxide.

47. A method of claim 41, wherein the mixed metal oxide has the empirical formula Mo₁VₐNb_{b}Sb_{c}Oₓ, and in the admixing step the compounds of Mo, V, Nb and Sb are added in relative molar amounts such that a ranges from about 0.1 to about 0.6, b ranges from about 0.02 to about 0.12, and c ranges from about 0.1 to about 0.5, and x depends on the oxidation state of other elements present in the final mixed metal oxide, or the empirical formula Mo₁VₐTa_{b}Sb_{c}Oₓ, and in the admixing step the compounds of Mo, V, Ta and Sb are added in relative molar amounts such that a ranges from about 0.1 to about 0.6, b ranges from about 0.02 to about 0.12, c ranges from about 0.1 to about 0.5, and x depends on the oxidation state of other elements present in the final mixed metal oxide.

48. A method of claim 41, wherein the mixed metal oxide further comprises germanium and the admixing step further comprises admixing a compound of Ge.

49. A method of claim 48, further comprising, after the recovery step, the steps of:
optionally washing the recovered mixed metal oxide;
drying the recovered missed metal oxide; and
calcining the recovered mixed metal oxide.

50. A method of claim 48, wherein the mixed metal oxide has the empirical formula Mo₁VₐNb_{b}Sb_{c}Ge_{d}Oₓ, and in the admixing step the compounds of Mo, V, Nb, Sb and Ge are added in relative molar amounts such that a ranges from about 0.1 to about 0.6, b ranges from about 0.02 to about 0.12, c ranges from about 0.1 to about 0.5, d ranges from about 0.01 to about 1, and x depends on the oxidation state of other elements present in the mixed metal oxide, or the empirical formula Mo₁VₐTa_{b}Sb_{c}Ge_{d}Oₓ, and in the admixing step the compounds of Mo, V, Ta, Sb and Ge are added in relative molar amounts such that a ranges from about 0.1 to about 0.6, b ranges from about 0.02 to about 0.12, c ranges from about 0.1 to about 0.5, d ranges from about 0.01 to about 1, and x depends on the oxidation state of other elements present in the final mixed metal oxide.

51. A method of claim 48, wherein d, in both empirical formulas, ranges from greater than 0.1 to about 1.

52. The method of claim 41 or of claims depending therefrom, wherein the agitation of the reaction medium during the reacting step is accomplished by stirring the reaction medium within the reaction vessel or by shaking, tumbling or oscillating the reaction vessel.

53. A catalyst comprising a mixed metal oxide effective for vapor phase conversion of propane to acrylic acid or acrylonitrile or isobutane to methacrylic acid or methacrylonitrile, the mixed metal oxide being prepared by the method of claim 29, 38, or of claims depending therefrom.

54. The method of claims 30, 41, or of claims depending therefrom wherein the temperature is at least about 125° C., and the pressure is at least about 25 psig.

55. The method of claim 51 wherein the temperature is at least about 150° C. and the pressure is at least about 50 psig.

56. The method of claim 51, wherein the temperature is at least about 175° C. and the pressure is at least about 100 psig.

57. The method of claims 30, 41 or of claims depending therefrom, wherein the mixed metal oxide precursor is calcined in an oxygen-containing atmosphere at a temperature of at least about 500° C. to form the mixed metal oxide.

58. A method of converting propane to acrylic acid, the method comprising:
providing the catalyst of claim 10, 19 or of claims depending therefrom in a gas-phase flow reactor, and
contacting the catalyst with propane in the reactor in the presence of oxygen under reaction conditions to form acrylic acid.

59. A method of converting of propane to acrylonitrile, the method comprising:
providing the catalyst of claim 10, 19 or of claims depending therefrom in a gas-phase flow reactor, and
contacting the catalyst with propane in the reactor in the presence of oxygen and ammonia under reaction conditions to form acrylonitrile.

60. The method of claim 59, wherein the catalyst is contacted with isobutane in the reactor in the presence of oxygen and ammonia under reaction conditions that include a temperature ranging from about 300° C. to about 550° C., and at a pressure ranging from about 0 psig to about 200 psig.

61. The method of claim 59, wherein the catalyst is contacted with propane in the reactor in the presence of oxygen and ammonia under reaction conditions that include a weight hourly space velocity (WHSV) ranging from about 0.02 to about 5.

62. A method of converting isobutane to methacrylic acid, the method comprising:
providing the catalyst of claim 10, 19 or of claims depending therefrom in a gas-phase flow reactor, and
contacting the catalyst with isobutane in the reactor in the presence of oxygen under reaction conditions to form methacrylic acid.

63. A method of converting of isobutane to methacrylonitrile, the method comprising:
providing the catalyst of claim 10, 19 or of claims depending therefrom, in a gas-phase flow reactor, and
contacting the catalyst with propane in the reactor in the presence of oxygen and ammonia under reaction conditions to form acrylonitrile.

64. The method of claim 59, wherein the catalyst is contacted with propane in the reactor in the presence of oxygen and ammonia under reaction conditions that include a temperature ranging from about 300° C. to about 550° C., and at a pressure ranging from about 0 psig to about 200 psig.

65. The method of claim 59, wherein the catalyst is contacted with isobutane in the reactor in the presence of oxygen and ammonia under reaction conditions that include a weight hourly space velocity (WHSV) ranging from about 0.02 to about 5.
